(19) United States
(12) Patent Application Publication
(10) Pub. No.: US 2015/0126907 A1
(43) Pub. Date: May 7, 2015

(54) SCALE AND ASSOCIATED METRIC FOR TREATMENT OF FACIAL WRINKLES AND RELATED CONDITIONS

(71) Applicant: LIVING PROOF, INC., Cambridge, MA (US)

(72) Inventors: Betty Yu, Cambridge, MA (US); Brian Biesman, Nashville, TN (US)

(21) Appl. No.: 14/527,983

(22) Filed: Oct. 30, 2014

Related U.S. Application Data
(60) Provisional application No. 61/897,528, filed on Oct. 30, 2013.

(51) Int. Cl. A61B 5/00

(52) U.S. Cl. CPC A61B 5/442; A61B 5/4878
USPC 600/587

(57) ABSTRACT

This invention provides assessment scale devices and methods for assessing the severity of wrinkles and facial swelling and puffiness. Wrinkle and facial swelling/puffiness assessment using the devices, methods, and assessment scales of the invention can be performed in a clinical setting for the purpose of simply characterizing a wrinkle or facial swelling/puffiness, or as part of a treatment plan or course that varies according to the severity of the wrinkle or facial swelling/puffiness. Thus, this invention also provides methods of reducing the appearance of wrinkles and/or facial puffiness, and kits comprising for evaluating and treating wrinkles and/or facial puffiness. Additionally, the present disclosure relates to a devices, methods, and assessment scales for managing expectations of the given treatment plan or course for human facial wrinkles and facial puffiness.

### RELATED APPLICATIONS

This application claims the benefit of U.S. Provisional Patent Application No. 61/897,528, filed Oct. 30, 2013, the entire contents of which are hereby incorporated by reference.

### FIELD OF THE DISCLOSURE

The present disclosure relates to a scale and metric for determining a treatment plan or course for a given combination of degree of human facial wrinkles and of degree of human facial puffiness or swelling. The present disclosure also relates to a scale and metric for determining a treatment plan or course for a degree of human facial puffiness or swelling. Additionally, the present disclosure relates to a scale and metric for managing expectations of the given treatment plan or course for human facial wrinkles and human facial puffiness or swelling. Additionally, the present disclosure relates to a scale and metric for managing expectations of the given treatment plan or course for human facial puffiness or swelling.

### BACKGROUND

Lower lid fat pad herniation is a prevalent problem associated with aging. Sun exposure and chronological skin aging processes result in a deterioration of the skin mechanical integrity. Along with the skin, the underlying orbital septum, which constrains the orbital fat, becomes slack and enables the fat pads to herniate. The severity of the herniation has not been characterized, to date, utilizing a standardized scale that describes the extent of fat pad herniation with a continuous or discrete grading system. While scales that grade other aesthetic features such as crow's feet, lip fullness, nasolabial wrinkle, forehead wrinkles and hands, no scale exists for objective quantitation of lower lid bag severity.

One dimensional scales for the assessment of human facial wrinkles are known. An example of a one dimensional measuring device for human facial wrinkles is disclosed in U.S. Published Patent Application No. 2013/0197397 A1 entitled “Methods and Assessment Scales for Measuring Wrinkle Severity”, published on Aug. 1, 2013 on behalf of Waugh and Blitzer. Similarly, a one dimensional clinical assessment scale is disclosed in U.S. Published Patent Application No. 2011/0160617 A9, entitled “Clinical Assessment Scales and Methods”, published on Jun. 30, 2011 on behalf of Thomas and Walker. Likewise, one dimensional scales and similar devices and methods for physical conditions are disclosed in U.S. Published Patent Application No. 2010/0121223 A1, entitled “Clinical Assessment Scales and Methods”, published on May 13, 2010 on behalf of Friborg et al.

However, none of the above scales are able to assess severity of eye puffiness or swelling (i.e., “eye bags”). Moreover, none of the scales can assess two distinct facial features concurrently. Finally, these references do not provide a treatment plan or course based upon the metric measured and in particular based on the combination of the degree of human facial wrinkles and the degree of human facial puffiness or swelling. Moreover, these references do not provide for a method which will manage the expectations of the patient as to the result of the treatment plan or course.

### OBJECTS AND SUMMARY OF THE DISCLOSURE

The present invention is a two dimensional scale or metric for assessing the severity of both wrinkles, for example, wrinkles on a human face, and swelling, for example, swelling under the eyes of a human subject. Other features that may be measured may include, for example, pigmentation, surface roughness and mechanical recoil. The scale of the present invention enables measurement of two distinct facial features at the same time. In addition, it is an object of the present disclosure to present a scale and metric to provide a treatment plan or course based on the combination of degree of human facial wrinkles and human facial puffiness or swelling.

One embodiment of the invention is an assessment device for quantifying severity of conditions associated with human facial wrinkles and swelling and for determining a treatment plan or treatment course. The assessment device includes, for example, a data collection of at least two coordinates, including at least a first coordinate and a second coordinate. The first coordinate includes a plurality of increments of degree of wrinkle severity. The second coordinate includes a plurality of increments of degree of facial swelling. The data collection includes a plurality of data spaces, wherein a data space is formed for each combination of one of the plurality of increments of degree of wrinkle severity and one of the plurality of increments of degree of facial swelling. Each of the data spaces includes data which corresponds to a treatment plan or treatment course for the combination of wrinkle severity and facial swelling associated with the data space.

Another embodiment of the invention is a method of treating wrinkles and facial swelling. The method includes the steps of: providing an assessment device which includes a plurality of data spaces which correspond to treatment plans or treatment course for combinations of degree of wrinkle severity and degree of facial swelling associated with the data space. Next, the assessment device is used to determine a degree of wrinkle severity and a degree of facial swelling of a patient. A treatment plan or treatment course is applied to the patient. The treatment plan or treatment course corresponds to the data space which corresponds to the determined degree of wrinkle severity and determined degree of facial swelling of the patient. The assessment device includes a data collection of at least two coordinates, including at least a first coordinate and a second coordinate. The first coordinate including a plurality of increments of degree of wrinkle severity. The second coordinate including a plurality of increments of degree of facial swelling. The data collection including the plurality of data spaces, wherein a data space is formed for each combination of one of the plurality of increments of degree of wrinkle severity and one of the plurality of increments of degree of facial swelling.

Another embodiment is an assessment device for quantifying severity of conditions associated with human facial wrinkles and swelling and for determining a treatment plan or treatment course. The assessment device includes a data collection of at least two coordinates, including at least a first coordinate and a second coordinate. The first coordinate being a range of wrinkle severity. The second coordinate being a range of facial swelling. The data collection including range of data corresponding to intersections of the first coordinate and second coordinate. The data in the data collection corresponds to a treatment plans or treatment courses for the wrinkle severity and facial swelling associated with an intersection of the first coordinate and the second coordinate.

The present invention also includes a scale or metric for assessing the swelling, for example, swelling under the eyes of a human subject. In addition, it is an object of the present disclosure to present a scale and metric to provide a treatment plan or course based on the degree of human facial puffiness or swelling. The appearance of puffiness or swelling may be caused by the herniation of the underlying fat pads.

It is therefore a further object of the present disclosure to provide a method to manage the expectations of the result of a treatment plan or course for human facial wrinkles and facial swelling or puffiness.

### BRIEF DESCRIPTION OF THE DRAWINGS

Further objects and advantages of the disclosure will become apparent from the following description and from the accompanying drawings, wherein:

FIG. 1 is an alpha-numeric lower lid photo scale of the present disclosure.

FIG. 2 is a lower lid alpha-numeric photo-typing grid of the present disclosure.

FIG. 3 is a lower lid alpha-numeric photo-typing grid of the present disclosure.

FIG. 4 includes a question which may be asked of patients, followed by a series of possible answers.

FIG. 5 is an alpha-numeric lower lid photo scale of the present disclosure.

FIG. 6 is an numeric lower lid photo scale of the present disclosure.

FIG. 7 is a chart comparing a scale of the present invention to the global aesthetic improvement scale (GAIS).

FIG. 8 is a chart comparing a scale of the present invention to the global aesthetic improvement scale (GAIS).

### DETAILED DESCRIPTION OF THE PREFERRED EMBODIMENTS

The present disclosure relates to a method and apparatus for assessment of human facial wrinkles and associated conditions, such as “puffiness” or swelling. Wrinkle and swelling assessment using the assessment scale devices and methods of the invention can be performed for the purpose of simply characterizing a wrinkle or swelling, or as part of a treatment regimen that varies according to the severity of the wrinkle or swelling. The present disclosure further provides a method and apparatus for formulation of at least one treatment plan or treatment course, and further provides an opportunity for managing the expectations of the patient with respect to the probable outcome of the treatment plan or treatment course.

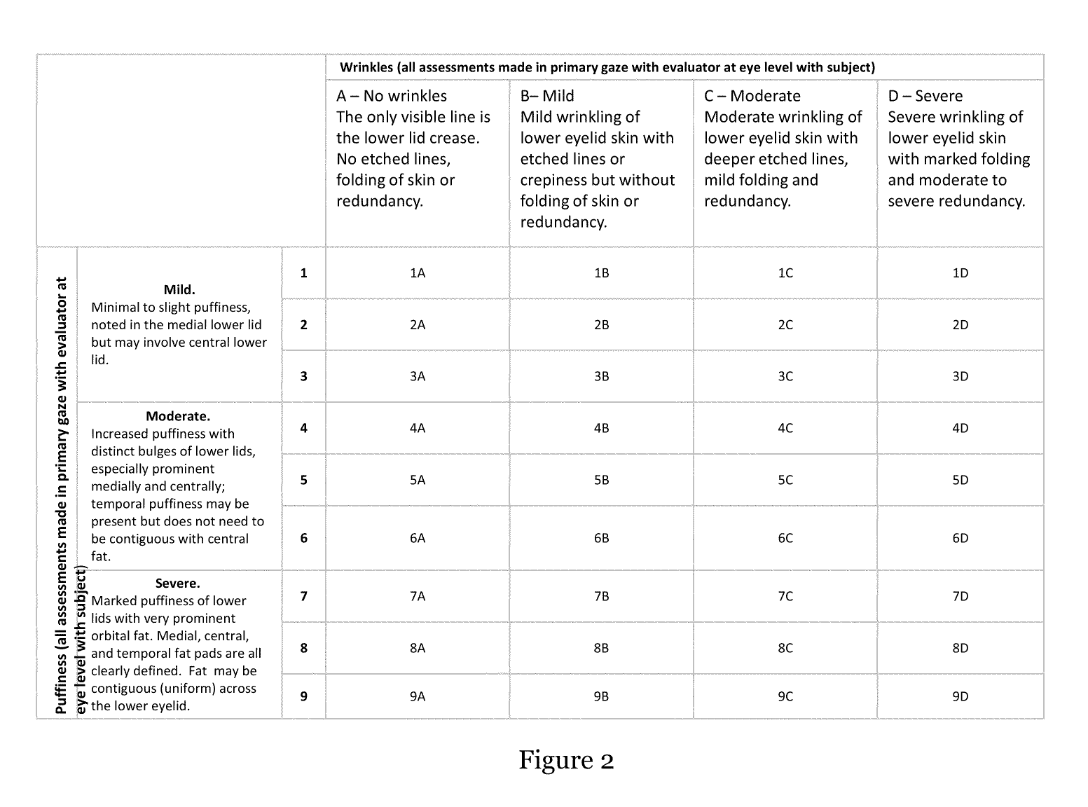

The present invention provides a lower lid bag tool enables a physician to diagnose the severity of the bag using a standardized lexicon, and set the patient expectations with respect to the anticipated outcome of the procedures recommended. For example, for severe bags (correlating to puffiness score of 7-9 on the scale as shown in FIGS. 2, 5, and 6) the patient may be a candidate for a lower lid blepharoplasty, but for mild bags (correlating to puffiness score of 1-3 on the scale as shown in FIGS. 2, 5, and 6) a patient may be better suited for lower lid filler injections. As a tool for setting expectations, the photo-numeric scale provides a reference for the doctor to describe the expected improvement. For example, a blepharoplasty for lower lids graded with a severity of 8 on the scales shown in FIGS. 2, 5, and 6 may result in the appearance of the image corresponding to a grade of 1. Similarly, the use of dermal fillers for a patient with a lower lid severity grade of 3 may yield an improvement represented by the photo corresponding to grade 1.

In the absence of such a standardized grading scale for bag severity, the diagnosis and treatment of bags relies on personal verbal communication from the physician to the patient. The doctor assesses the bag severity based on a subjective database of patient severities and the corresponding efficaciousness of the treatment outcome. Without a quantitative grading scale, the magnitude of the problem as well as the extent of the treatment efficacy poses challenges to benchmarking progress as well as deterioration of the lower lid state. The ability to quantify the problem on an absolute scale enables comparison and differentiation of bag severities to define the efficacy of each recommended treatment modality specific to each bag grade.

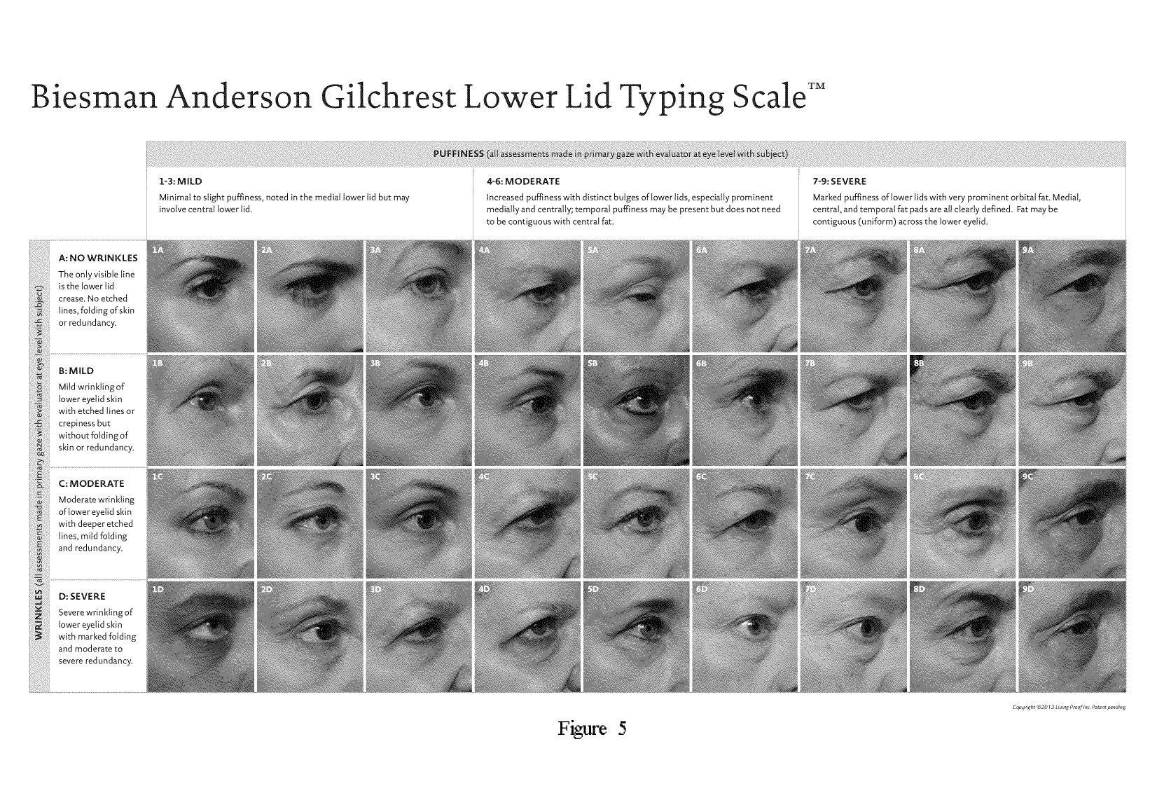

The wrinkle scales existing in the literature describe skin folds utilizing two descriptors to capture the wrinkle size and the depth of the wrinkle, but the wrinkles are not associated with changes in the volume of the underlying structures. The present invention as described herein is a two dimensional scale that allows for measurement of two distinct facial features (for example, wrinkles and lower lid bags). For example, a standard wrinkle scale may assign four categories (none, mild, moderate and severe) which translates to 4 categories. The scale presented in FIGS. 2 and 5 captures 36 different lower lid severities, because nine levels of under eye puffiness/swelling have also be categorized and integrated into the wrinkle scale. The integration of the two measurements on one scale provides several benefits, including ease of use; improved accuracy; and a more complete diagnosis of the patient's condition. For example, rather than being limited to describing wrinkles only, the present invention can capture the wrinkle severity in addition to the underlying bag size. The benefit of using the assessment devices of the present invention enables a complete diagnosis of the lower lid aesthetic condition to capture the contribution of the fat pads to the overall aesthetic score, which would in turn, properly reflect the recommended treatment modalities available to improve the patient appearance.

The present invention uses a standardized lexicon that provides a uniform baseline from which the user can make the measurement, therefore reducing user bias and user-to-user variation. An additional benefit of the standardized lexicon of the present invention is that it enables the physician to track the patient changes over the course of care or refer the patient to another physician without creating gap in medical history.

### Assessment Scale Devices

One embodiment of the invention is an assessment device for quantifying severity of conditions associated with wrinkles (for example, facial wrinkles) and swelling or puffiness (for example, under-eye bags) and for determining a treatment plan or treatment course. The assessment device includes a data collection of at least two coordinates, including at least a first coordinate and a second coordinate. The first coordinate includes a plurality of increments of degree of wrinkle severity. The second coordinate includes a plurality of increments of degree of facial swelling. The data collection includes a plurality of data spaces, wherein a data space is formed for each combination of one of the plurality of increments of degree of wrinkle severity and one of the plurality of increments of degree of facial swelling. Each of the data spaces includes data which corresponds to a treatment plan or treatment course for the combination of wrinkle severity and facial swelling associated with the data space. The outcome of the treatment (i.e., the projected new wrinkle severity and eye swelling/puffiness score for the patient) may be included in the device.

Another embodiment of the invention is an assessment device for quantifying swelling or puffiness (for example, under-eye bags) and for determining a treatment plan or treatment course. The assessment device includes a data collection of at least one coordinate, including at least a first coordinate. The first coordinate includes a plurality of increments of degree of facial swelling as viewed from the front of, from 45° from front, and from a profile of the subject. The data collection includes a plurality of data spaces, wherein a data space is formed for plurality of increments of degree of facial swelling. Each of the data spaces includes data which corresponds to a treatment plan or treatment course for the facial swelling associated with the data space. The outcome of the treatment (i.e., the projected eye swelling/puffiness score for the patient) may be included in the device.

One embodiment of this invention is an assessment device for quantifying severity of conditions associated with human facial wrinkles and swelling and for determining a treatment plan or treatment course, the assessment device comprising: at least two coordinates, including at least a first coordinate and a second coordinate; the first coordinate including a plurality of increments of degree of wrinkle severity; the second coordinate including a plurality of increments of degree of facial swelling; wherein a data space is formed for each combination of one of the plurality of increments of degree of wrinkle severity and one of the plurality of increments of degree of facial swelling; and each of the data spaces including data which corresponds to a treatment plan or treatment course for the combination of wrinkle severity and facial swelling associated with the data space.

One embodiment of this invention is an assessment device for quantifying severity of conditions associated with human facial swelling and for determining a treatment plan or treatment course, the assessment device comprising: a plurality of increments of degree of facial swelling; wherein a data space is formed for the plurality of increments of degree of facial swelling; and each of the data spaces including data which corresponds to a treatment plan or treatment course for the facial swelling associated with the data space.

The term “wrinkle”, as used herein, refers to a fold or crease in the skin, which vary both in size and intensity. For example, a wrinkle may be a fine lines to or a deep furrows, or any fold or crease in between. Skin wrinkles may be classified into three different types: dynamic wrinkles, static wrinkles and wrinkle folds. Repeated contractions of muscles underlying the skin cause dynamic wrinkles For example, frowning or furrowing causes wrinkles between the eyebrows (i.e., glabellar lines), while smiling and/or squinting causes wrinkles at the distal corners of the eyes (i.e., lateral canthal lines). Static wrinkles, or wrinkles at rest, when the face is in a neutral or natural position, result from a loss of elasticity in skin, which may arise from a variety of factors, including sun damage, chronologic aging, poor nutrition, smoking, and genetic factors, or from spasms or tones of muscles. Wrinkle folds, which may appear as deep grooves between the nose and mouth, for example, arise from the deterioration of underlying facial structure, such as loss of facial volume, elastosis, and collagen degradation.

The assessment scale devices and methods of this invention are suitable for measurement and assessment of wrinkle severity of all types of wrinkles, which may be present on any area of skin of a subject in need of treatment. In addition to the face and neck, non-limiting examples of areas that also may be treated include the hands, arms, feet, legs, shoulders, chest, torso and back. In addition, when the area to be evaluated is the face, the wrinkles may be located in specific subregions of the face, such as the forehead, eyes, temples, cheeks, jawline or nose. This invention also provides for treatment plan or treatment course based on the degree of wrinkle severity as determined according to the invention.

Additionally, the assessment scales and methods of this invention are suitable for measurement and assessment of swelling or puffiness. Swelling or puffiness means the enlargement of a body part or area not caused by proliferation of cells. This invention contemplates swelling due to any cause, but in particular, swelling caused by fluid retention. Swelling and puffiness are used interchangeably throughout this application and are intended to have the same meaning. In particular, the assessment scales and methods can be used to assess eye bags, or more particularly, under-eye bags. The term “eye bag”, as used herein, refers to a puffiness or swelling under the eye. The term “eye bags” (also known as “periorbital puffiness” or “puffy eyes” or swelling around the eyes), refers to the appearance of swelling in the tissues around the eyes (i.e., the orbits). Eye bags may be caused by fluid buildup around the eyes, or periorbital edema. Eye bags are typically present under the eye, may also be present all around the eye. Eye bags may be transient or age-related. Causes of eye bags include infection, sleep deprivation, fluid retention, diet, alcohol and/or drug use, allergies, skin disorders (including dermatitis), crying or the gradual increase in the size of the fat pad lying below the lower eye lids. Eye bags can vary in size and intensity, from mild to moderate to severe puffiness or swelling.

Generally, the assessment scales of the invention comprise a plurality of levels, where each level corresponds to a different degree of wrinkle severity or swelling/puffiness. “Data collection” refers to the ascertaining the severity of wrinkle or swelling within these plurality of levels. The data collection includes a plurality of data spaces, wherein a data space is formed for each combination of one of the plurality of increments of degree of wrinkle severity and one of the plurality of increments of degree of swelling/puffiness. In other words, the data space is the resultant value for a patient with a particular wrinkle severity and a particular amount of swelling. In certain embodiments, the levels of the assessment scale are defined by reference to one or more physical characteristics of the wrinkle, non-limiting examples of which include length, width, depth, area, morphology, position, skin rigidity, volume, shape of underlying muscle, quantity of wrinkles, and wrinkle-to-wrinkle distance and one or more physical characteristics of the swelling/puffiness, non-limiting examples of which include amount of swelling/puffiness, location of the swelling/puffiness, appears of bulges and/or definition of underlying fat pad. Each level in the assessment scale may be distinguished from the others based on descriptors that relate to measured physical characteristics. The form of the descriptors is not particularly limited and may comprise text, images, or combinations thereof. Optionally, a rating system may be used to uniquely identify each level of the assessment scale. For instance, the rating system may be numerical, with the lowest number of the rating system corresponding to the least severe level of wrinkles or eye swelling/puffiness, and the highest number of the rating system corresponding to the most severe level of wrinkles or eye swelling/puffiness. Each of the data spaces includes data which corresponds to a treatment plan or treatment course for the combination of wrinkle severity and facial swelling associated with the data space.

Alternatively, the assessment scales of the invention comprise a plurality of levels, where each level corresponds to a different degree of swelling/puffiness. “Data collection” refers to the ascertaining the severity swelling within these plurality of levels. The data collection includes a plurality of data spaces, wherein a data space is formed for the plurality of increments of degree of swelling/puffiness. In other words, the data space is the resultant value for a patient with a particular amount of swelling. In certain embodiments, the levels of the assessment scale are defined by reference to one or more physical characteristics of the swelling/puffiness, non-limiting examples of which include amount of swelling/puffiness, location of the swelling/puffiness, appears of bulges and/or definition of underlying fat pad. Each level in the assessment scale may be distinguished from the others based on descriptors that relate to measured physical characteristics. The form of the descriptors is not particularly limited and may comprise text, images, or combinations thereof. Optionally, a rating system may be used to uniquely identify each level of the assessment scale. For instance, the rating system may be numerical, with the lowest number of the rating system corresponding to the least severe level of eye swelling/puffiness, and the highest number of the rating system corresponding to the most severe level of eye swelling/puffiness. Each of the data spaces includes data which corresponds to a treatment plan or treatment course for the facial swelling associated with the data space.

In another embodiment, the invention is an assessment scale device for quantifying severity of conditions associated with wrinkles and swelling/puffiness and for determining a treatment plan or treatment course. In certain aspects, this embodiment includes the assessment device including a data collection of at least two coordinates, including at least a first coordinate and a second coordinate. The first coordinate is a range of wrinkle severity and the second coordinate being a range of facial swelling. The data collection includes a range of data corresponding to intersections of the first coordinate and second coordinate. The data in the data collection corresponds to a treatment plans or treatment courses for the wrinkle severity and swelling associated with an intersection of the first coordinate and the second coordinate.

In another embodiment, the invention is an assessment scale device for quantifying severity of conditions associated with swelling/puffiness and for determining a treatment plan or treatment course. In certain aspects, this embodiment includes the assessment device including a data collection of at least one coordinate, including at least a first coordinate. The first coordinate is a range of facial swelling. The data in the data collection corresponds to a treatment plans or treatment courses for swelling.

A coordinate refers to the value determined or measured for either wrinkle severity or eye swelling. The value may be numerical or in qualitative words (e.g., mild, moderate or severe). In one aspect, the first coordinate includes a plurality of descriptions of the increments of degree of wrinkle severity. In one aspect, the second coordinate includes a plurality of descriptions of the increments of degree of facial swelling. In some embodiments, at least one of the increments of degree of facial swelling includes a plurality of sub-increments, wherein each of the sub-increments includes a description of the sub-increment of facial swelling. In some embodiments, at least one of the increments of degree of wrinkle severity includes a plurality of sub-increments, wherein each of the sub-increments includes a description of the sub-increment of wrinkle severity. Each combination of a sub-increment of degree of facial swelling and increment of degree of wrinkle severity corresponds to one of the data spaces. Alternatively, the coordinates are a range of continuous values measured independently (i.e., as wrinkle severity and swelling) and the data collection is the intersection of these two coordinates. For example, the continuum may be a photograph of the subject's face which can be simultaneously modified to depict the degree of wrinkles and swelling both before and after treatment. Alternatively, the continuum may be the data space spanned by four coordinates explicitly defined by the measurement device, where non-discrete values are assigned within this space by the evaluator.

The form of the assessment scale is not limited, and may be in any format suitable for storing and organizing information. In certain embodiments, the assessment scale is depicted as a table, where each row of the table corresponds to a different level of severity. An assessment device includes any suitable format for displaying the first and second coordinate. The assessment device is in tabular form. In some embodiments, the data collection is organized into rows and columns. For example, the assessment device may be a grid or chart wherein the first and second coordinate are arranged into two different lines at right angles to one another. In some embodiments, the assessment device further includes depictions corresponding to each increment of wrinkle severity and each increment of facial swelling. In a particular embodiment, the depictions are photographs. The photographs may be of the same subject taken from different views (e.g., from front, from 45° from front, or from 90° from front (i.e., a profile view)). Alternatively, the first coordinate and second coordinates may be displayed on two concentric wheels that are attached but can be moved independent of each other. When the assessment device is in this format, the user moves each wheel independently to identify the assessment or measurement for the first and second coordinate. Once each wheel has been rotated to identify the measurement, a window on the assessment device may be opened to identify the treatment plan or course. In certain embodiments, the assessment device is stored in electronic form, such as on a computer readable medium or in a database (e.g., a relational database), and accessed as needed. Additionally, it is envisioned that the vertical axis and the horizontal axis could be continuous and could be implemented so that a computer pointing object (such as, but not limited to, a mouse, trackball or even a finger) could be used to select points along a two-dimensional area or multi-dimensional space, thereby choosing or formulating a treatment plan or course. It is likewise envisioned that the photo-scale could be implemented as a series of tangible sheets of information, which may be in tabular form, or in any number of database implementations, including, but not limited to, cartesian, spherical or cylindrical coordinates.

The treatment plan or treatment course corresponds to the data space which corresponds to the determined degree of wrinkle severity and determined degree of facial swelling of the patient. Alternatively, the treatment plan or treatment course corresponds to the data space which corresponds to the determined degree of facial swelling of the patient. The treatment plans or treatment courses further include data as to at least one expected outcome of the treatment plan or treatment course. A treatment plan or treatment course means the regimen for treating the wrinkle and/or swelling. Following a treatment plan or treatment course should decrease the degree of wrinkle severity or eye swelling. Current methods for treating wrinkles and eye swelling include invasive and non-invasive methods and formulations. Invasive techniques, include surgery, fillers (e.g., Restylane, Juvederm), laser resurfacing or Botox®. Examples of non-invasive methods include treating wrinkles or eye swelling by applying a foundation-type make-up to the skin or applying a cosmetic formulation that includes an ingredient that may reduce the appearance of the imperfections over time (e.g., an anti-wrinkle cream). Anti-wrinkle compositions contemplated by the invention may also comprise other anti-wrinkle agents known in the art, non-limiting examples of which include retinol, alpha-hydroxy acid, collagen, elastin, and hyaluronic acid. Alternatively, the treatment plan or course maybe use of polymeric films, such as those described in US Application No. 2012/0251600 and US 2013/0078209, the teachings of which are incorporated herein by reference. Reassessment of a patient's wrinkle severity and eye swelling/puffiness should result in the reduction of the appearance of wrinkles and eye bags.

Assessments of the degree of wrinkle severity and the degree of facial swelling are made in a primary gaze of a subject with the evaluator at eye level with the subject.

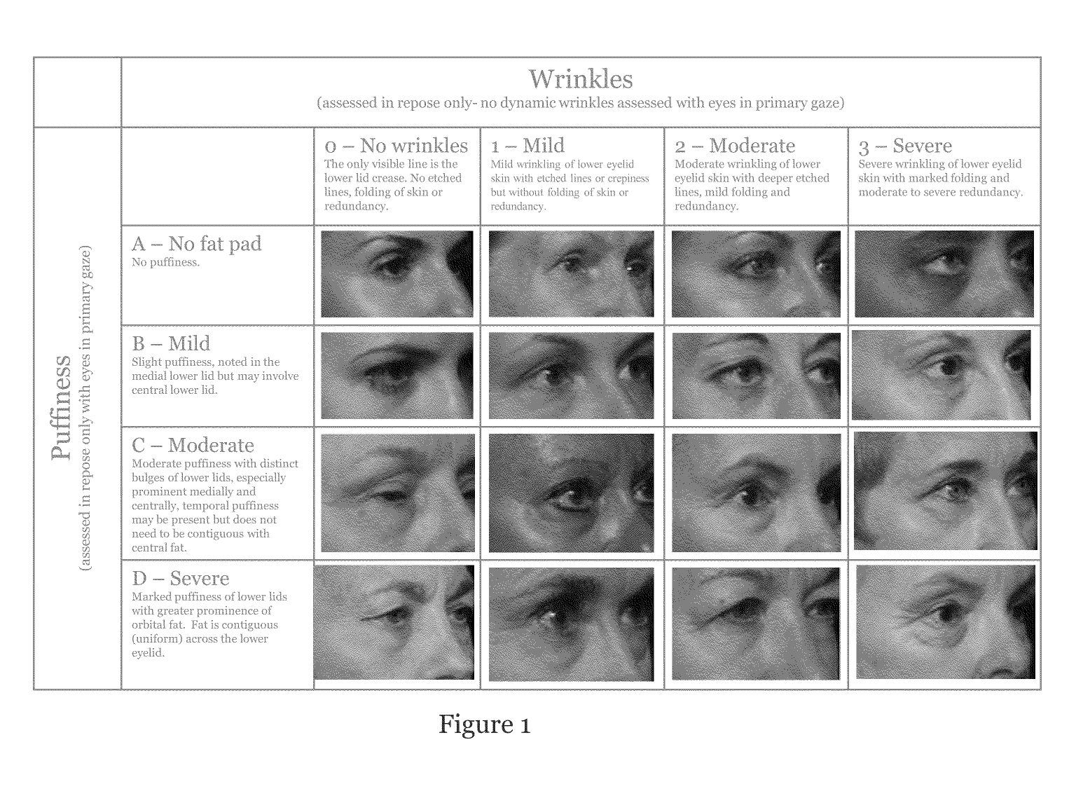

Examples of various embodiments of the invention are presented in FIGS. 1-8.

FIG. 1 gives an example of a scale with the photographs incorporated directly into the scale.

FIG. 2 is an example of an alpha-numeric lower lid photo-scale.

The vertical axis indicates increments of degree of “puffiness” or swelling. There are three major categories of degree of “puffiness” or swelling —“mild”, “moderate” and “severe”, each of which are further sub-divided into three sub-increments of degree of “puffiness” or swelling so that the illustrated photo-scale includes nine different sub-increments of “puffiness” or “swelling”. The category of “mild” with respect to swelling typically means that there is minimal to slight puffiness, noted in the medial lower lid but may involve central lower lid. The category of “moderate” with respect to swelling typically means that there is increased puffiness with distinct bulges of lower lids, especially prominent medially and centrally; temporal puffiness may be present but does not need to be contiguous with central fat. The category of “severe” with respect to swelling typically means that there is marked puffiness of lower lids with very prominent orbital fat. Medial, central, and temporal fat pads are all clearly defined. Fat may be contiguous (uniform) across the lower eyelid. All assessments of degrees of puffiness or swelling are made in primary gaze with the evaluator at eye level with the subject.

The horizontal axis indicates increments of degree of facial wrinkles There are four categories of degree of facial wrinkles—“none”, “mild”, “moderate”, and “severe”. In further detail, the category of “none” with respect to wrinkles typically means that the only visible line is the lower lid crease. No etched lines, folding of skin or redundancy. The category of “mild” with respect to wrinkles typically means that there is mild wrinkling of lower eyelid skin with etched lines or crepiness but without folding of skin or redundancy. The category of “moderate” with respect to wrinkles typically means that there is moderate wrinkling of lower eyelid skin with deeper etched lines, mild folding and redundancy. The category of “severe” with respect to wrinkles typically means that there is severe wrinkling of lower eyelid skin with marked folding and moderate to severe redundancy. All assessments of degrees of facial wrinkling are made in primary gaze with the evaluator at eye level with the subject.

With this information, a grid with thirty-six squares is formed (nine increments or sub-increments of degree of swelling or “puffiness” by four increments of degree of wrinkling) The codes in squares are all two character codes, with the first character being chosen from the digits 1-9, corresponding to respective sub-increments of puffiness or swelling and the second character being chosen from the letters A-D, corresponding to the respective increments of degree of facial wrinkles Thus, the distinct readings for wrinkles and under-eye bags are correlated together within the same device, facilitating development of a treatment plan. For example, a patient with severe puffiness, but no wrinkles would be classified as a “0D” in the device shown in FIG. 1 and may require a different treatment plan than a patient with moderate puffiness and moderate wrinkles (for example, a 5C classification).

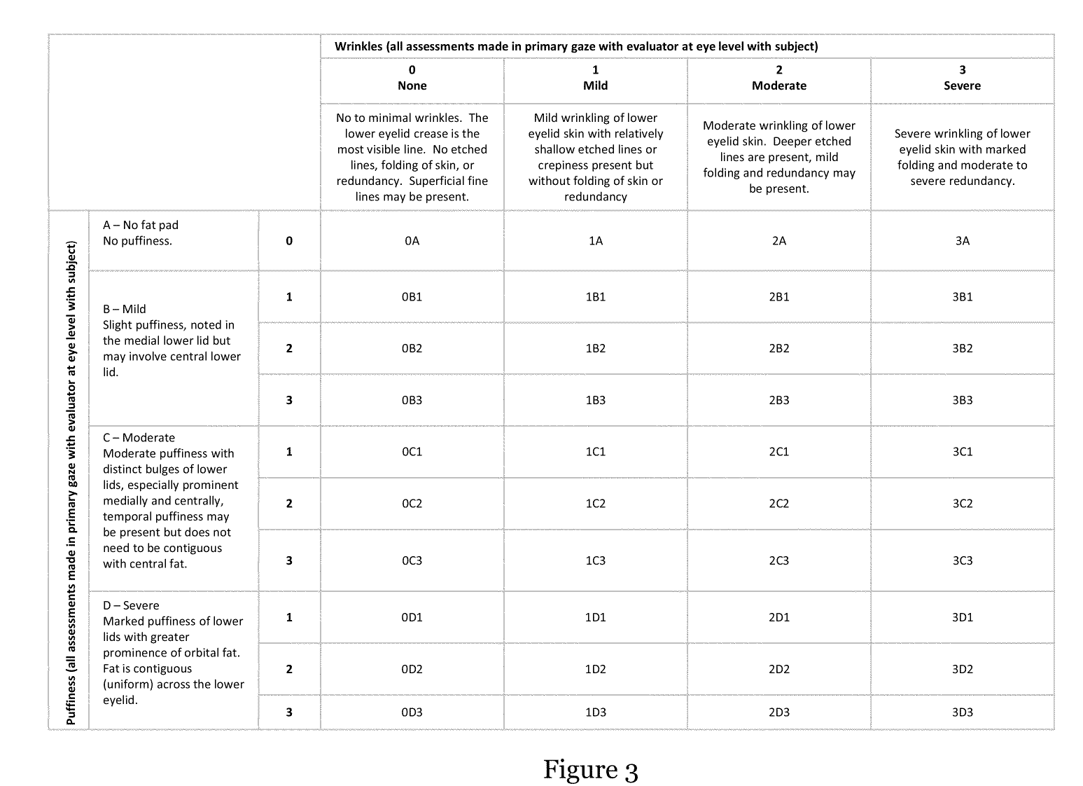

FIG. 3 is an alpha-numeric lower lid photo-scale which is similar to the scale disclosed in FIG. 2. With respect to FIG. 2, the category of “none” with respect to puffiness or swelling has been added. Thus, there are four major categories of degree of puffiness or swelling—“none”, “mild”, “moderate” and “severe”. The “mild”, “moderate” and “severe” categories are further sub-divided into three sub-increments of degree of puffiness or swelling so that the illustrated photo-scale includes ten different sub-increments of puffiness or swelling. In further detail, the category of “none” with respect to swelling typically means that there is no visible fat, pad or lower lid puffiness. The category of “mild” with respect to swelling typically means that there is slight puffiness, noted in the medial lower and/or central lower eyelid. The category of “moderate” with respect to swelling typically means that there is moderate puffiness with distinct bulges of lower lids, especially prominent medially and centrally, temporal puffiness may be present but does not need to be contiguous with central fat. The category of “severe” with respect to swelling typically means that there is marked puffiness of lower lids with greater prominence of orbital fat. Fat is contiguous (uniform) across the lower eyelid. All assessments of degrees of puffiness or swelling are made in primary gaze with the evaluator at eye level with the subject.

The given definitions of degree of swelling and degree of facial wrinkles on axes have been changed slightly in presentation, but are intended to be substantively unchanged. As shown in FIG. 3, each of the forty squares has a three-character code (simplified to a two-character code in the absence of swelling or “puffiness”).The first character indicates the degree of wrinkles—“0” for “none” 40, “1” for “mild”, “2” for “moderate” or “3” for severe. The second character indicates the degree of “puffiness” or swelling—“A” for “none”, “B” for “mild”, “C” for “moderate”, and “D” for “severe”. In the cases degrees of “puffiness” or swelling which are “mild”, “moderate” or “severe”, a third character is added for the sub-increment.

Each of the given two or three character codes corresponds to at least one treatment plan or treatment course which is determined in advance to provide at least one treatment plan or course to the medical practitioner after the diagnosis (i.e., the determination of the two or three character code) has been made.

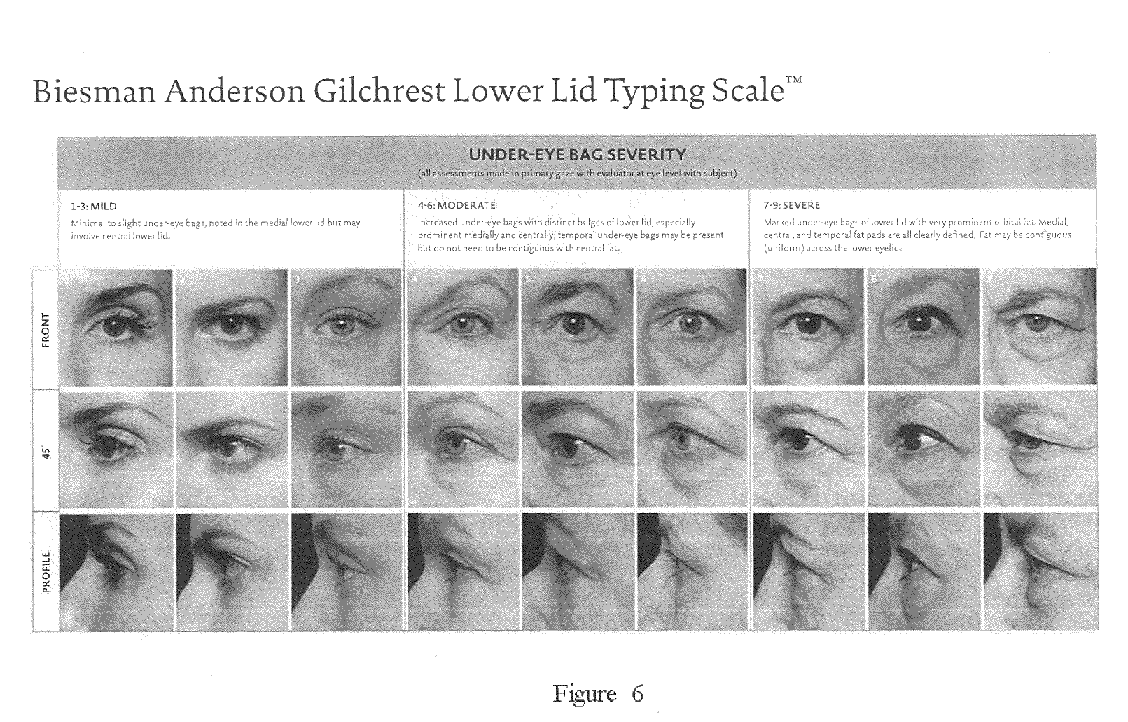

FIGS. 5 and 6 give additional examples of a scales with the photographs incorporated directly into the scale.

In FIG. 5, the horizontal axis indicates increments of degree of “puffiness” or swelling. As described above for FIG. 2, there are three major categories of degree of “puffiness” or swelling—“mild”, “moderate” and “severe”, each of which are further sub-divided into three sub-increments of degree of “puffiness” or swelling so that the illustrated photo-scale includes nine different sub-increments of “puffiness” or “swelling”. The vertical axis indicates increments of degree of facial wrinkles There are four categories of degree of facial wrinkles—“no wrinkles”, “mild”, “moderate”, and “severe”, as described above for FIG. 2. All assessments of degrees of puffiness or swelling are made in primary gaze with the evaluator at eye level with the subject. With this information, a grid with thirty-six squares is formed (nine increments or sub-increments of degree of swelling or “puffiness” by four increments of degree of wrinkling) The codes in squares are all two character codes, with the first character being chosen from the digits 1-9, corresponding to respective sub-increments of puffiness or swelling and the second character being chosen from the letters A-D, corresponding to the respective increments of degree of facial wrinkles

In FIG. 6, the horizontal axis indicates increments of degree of “puffiness” or swelling. As described above for FIG. 2, there are three major categories of degree of “puffiness” or swelling—“mild”, “moderate” and “severe”, each of which are further sub-divided into three sub-increments of degree of “puffiness” or swelling so that the illustrated photo-scale includes nine different sub-increments of “puffiness” or “swelling”. The vertical axis indicates the view of the subject: front (i.e., appearance when viewing directly into subject's eyes); 45° (i.e., appearance when viewing subject from a 45° from front); and profile (i.e., appearance when viewing subject from a 90° from front). All assessments of degrees of puffiness or swelling are made in primary gaze with the evaluator at eye level with the subject. With this information, a grid with twenty-seven squares is formed (nine increments or sub-increments of degree of swelling or “puffiness” by three increments of angle of view). The codes in squares are all one character codes, with the character being chosen from the digits 1-9, corresponding to respective sub-increments of puffiness or swelling.

It is envisioned that more than one treatment plan or course could be offered for a patient with a given diagnosis (i.e., with a determined two or three character code as shown in FIGS. 2 and 3 above or with a determined one character code as shown in FIG. 6 above). This would allow the medical practitioner to discuss treatment options which may involve different levels of treatment, which would typically correspond to different numbers of treatment sessions, different choices of procedures, different expenses, and different expectations as to the final result.

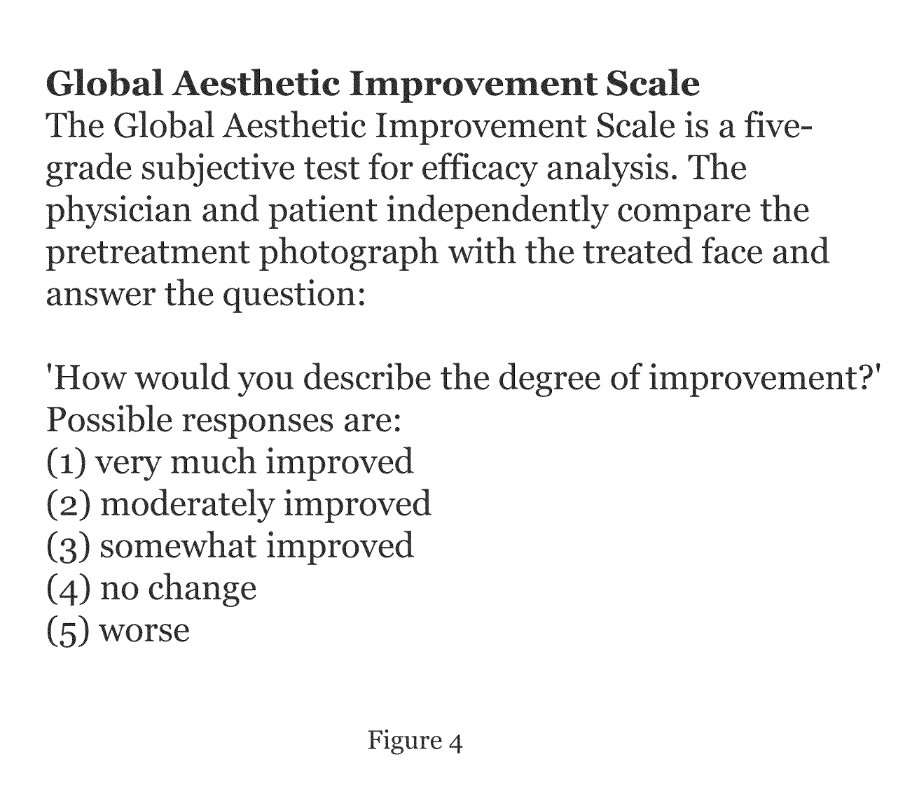

With respect to managing expectations as to the final result, the medical practitioner may be able to start with the diagnosis of the patient (in the form of the two or three digit code or a one character code), discuss and select a treatment plan or course with the patient, and indicate a final result, in the form of a two or three digit code (or alternatively a one character code) as used for the diagnosis, which may be a reasonable expectation for the patient after the treatment plan or treatment course. Additionally, it is envisioned that a medical practitioner may be able to take a photograph of the patient before treatment and through the use of imaging software, produce a picture for the patient which is representative of the patient's appearance after a selected treatment plan or course. This may also be used to compare the results of a number of different treatment plans or courses. This may also be implemented on a computer with a computer pointing device so that the medical practitioner, or even the patient, may view the various possible results. This could also be implemented to illustrate the expected age progression of the patient's appearance both with and without the various proposed treatment plans or courses. Additionally, a treatment course may be adjusted during the treatment course in response to the efficacy of the various steps and the patient's response thereto. The global aesthetic improvement scale of FIG. 4 provides a way for a patient to give a specific opinion with respect to the interim results, which can be used, either through the judgment of the medical practitioner or through the diagnosis software (or a combination of both) as to how the treatment course should be adjusted, or if it should be maintained.

In one embodiment, the invention provides an assessment scale for assessing the severity of a wrinkle or swelling or puffiness. In certain embodiments, the invention provides a wrinkle and facial swelling/puffiness assessment scale that is reliable, construct valid, and able to indicate progress following treatment. In certain embodiments, the invention provides a facial swelling/puffiness assessment scale that is reliable, construct valid, and able to indicate progress following treatment. A “reliable” assessment scale is one where the data collection is reproducibly measured. A “construct valid” assessment scale actually and reproducibly measures what it is designed to measure. Ways to determine whether the assessment scale is reliable and construct valid are known to one of skill in the art. An assessment scale is deemed “able to indicate progress following treatment” if the chosen measured parameter associated with the assessment scale permit a user to significantly and consistently distinguish changes due to the treatment plan or treatment course.

### Methods

The invention also provides a method for reducing the appearance of wrinkles and swelling in a subject. The method includes determining the wrinkle severity and facial swelling according to an assessment scale, and treating the wrinkle and/or swelling in accordance with a treatment that corresponds to the level of severity of the wrinkle or swelling to reduce the appearance of the wrinkle or swelling.

In another embodiment, the invention includes a method of treating wrinkles and facial swelling. The method includes providing an assessment device as described above and using the assessment device to determine a degree of wrinkle severity and a degree of facial swelling of a patient. The method further includes applying a treatment plan or treatment course to the patient. In certain embodiments, the method of assessing the degree of wrinkle severity and the degree of facial swelling are made in a primary gaze of a subject with the evaluator at eye level with the subject.

In yet another aspect, the invention provides a method for assessing wrinkle severity and facial swelling. The method includes a step of measuring at least one physical characteristic of a wrinkle and assessing wrinkle severity according to an assessment scale and measuring at least one physical characteristic of facial swelling/puffiness and assessing facial swelling/puffiness severity according to an assessment scale.

The invention also provides a method for reducing the appearance of wrinkles or facial swelling/puffiness in a subject. The method comprises assessing a level of severity of a wrinkle or facial swelling, and treating the wrinkle with a treatment that corresponds to the determined level of severity. In certain embodiments, wrinkle or facial swelling is assessed as part of an initial evaluation. Following the initial evaluation, a treatment protocol may be chosen according to the severity level of the wrinkle or facial swelling. The methods described herein for assessing wrinkle severity or facial swelling/puffiness also may be used for sequential measurements, in order to assess progress and/or the outcome of the treatment over a period of time. The period of time may be, without limitation, any time sufficient to detect changes in the physical characteristics of the wrinkle, such as a change in wrinkle length or wrinkle depth, or swelling, such as prominence of eye bag. In certain embodiments, the time period may be one day, three days, one week, two weeks, three weeks, four weeks, six weeks, or eight weeks, six months, a year or according to a schedule established by anyone assessing the progress of treatment, including for example, the individual undergoing treatment, the physician or other health care professional.

In certain embodiments, once the level of severity of a wrinkle or facial swelling is determined, a treatment corresponding to the determined level of severity is administered. Generally, treatment involves administering an effective amount of an anti-wrinkle composition and or anti-swelling compositions. The term “effective amount” as used herein means an amount of a composition that is sufficient to produce the desired effects, but that is implicitly safe amount (i.e. one that is low enough to avoid serious side effects). Desired effects include, but are not limited to, the attenuation of a physical characteristic of a wrinkle, such as a reduction in wrinkle length or wrinkle depth, for example, and reduction of swelling or puffiness of the face.

The invention also provides a method for reducing swelling in a subject. The method includes determining the facial swelling according to an assessment scale, and treating swelling in accordance with a treatment that corresponds to the level of severity of the swelling to reduce the appearance of the swelling.

In another embodiment, the invention includes a method of treating facial swelling. The method includes providing an assessment device as described above and using the assessment device to determine a degree of facial swelling of a patient. The method further includes applying a treatment plan or treatment course to the patient. In certain embodiments, the method of assessing the degree of facial swelling are made in a primary gaze of a subject with the evaluator at eye level with the subject.

In yet another aspect, the invention provides a method for assessing facial swelling. The method includes a step of measuring at least one physical characteristic of facial swelling/puffiness and assessing facial swelling/puffiness severity according to an assessment scale.

The invention also provides a method for reducing the appearance of facial swelling/puffiness in a subject. The method comprises assessing a level of severity of facial swelling, and treating with a treatment that corresponds to the determined level of severity. In certain embodiments, facial swelling is assessed as part of an initial evaluation. Following the initial evaluation, a treatment protocol may be chosen according to the severity level of the facial swelling. The methods described herein for assessing facial swelling/puffiness also may be used for sequential measurements, in order to assess progress and/or the outcome of the treatment over a period of time. The period of time may be, without limitation, any time sufficient to detect changes in the physical characteristics of the swelling, such as prominence of eye bag. In certain embodiments, the time period may be one day, three days, one week, two weeks, three weeks, four weeks, six weeks, or eight weeks, six months, a year or according to a schedule established by anyone assessing the progress of treatment, including for example, the individual undergoing treatment, the physician or other health care professional.

In certain embodiments, once the level of severity of facial swelling is determined, a treatment corresponding to the determined level of severity is administered. Generally, treatment involves administering an effective amount of an anti-swelling compositions. The term “effective amount” as used herein means an amount of a composition that is sufficient to produce the desired effects, but that is implicitly safe amount (i.e. one that is low enough to avoid serious side effects). Desired effects include, but are not limited to, reduction of swelling or puffiness of the face.

### Kits

In one aspect, the invention also provides a kit for assessing wrinkles and facial swelling. In another aspect, the invention also provides a kit for assessing facial swelling. The kit includes an assessment scale, which, without limitation, may be present in the kit as a table, chart, or as a data file on a computer-readable medium. The assessment scale comprises a plurality of levels, with each level corresponding to a different degree of wrinkle severity or facial swelling, as described herein. Optionally, the kit may include an anti-wrinkle or anti-swelling composition or a series of anti-wrinkle or anti-swelling compositions, which may be administered according to a level of wrinkle or swelling severity determined using the assessment scale.

Examples of representative treatment are given below.

### EXAMPLE 1

Fifteen patients with a range of lower lid puffiness and wrinkle severities were evaluated at baseline. A polymer film was then applied at the lower lid skin sites. Three trained observers (one ocularplastic surgeon and two nurses) assigned lower lid severity grades to each lower lid region for each patient, utilizing one embodiment of an alpha-photonumeric eye-typing scale. In this embodiment, 16 grades are available (see FIG. 1), where each grade is derived from the combination of a lower lid puffiness grade (A, B, C or D) and a lower lid wrinkle severity grade (0, 1, 2 or 3), where A and 0 corresponding to no visible puffiness and no visible wrinkles D and 3 correspond to severe puffiness and severe wrinkles at the lower lid. The results are provided in Table 1.

TABLE 1
Baseline lower lid severity grades utilizing
the alpha-photonumeric eye-typing scale.

| | Patient Left | Patient Right | Patient Left | Patient Right |
|---|---|---|---|---|
| | Puffiness | Puffiness | Wrinkle | Wrinkle |
| patient no. | Score | Score | Score | Score |
| 1 | D | D | 2 | 2 |
| 2 | D | D | 2 | 2 |
| 3 | D | D | 1 | 1 |
| 4 | D | D | 3 | 3 |
| 5 | C | B | 2 | 2 |
| 6 | C | C | 2 | 2 |
| 7 | C | C | 3 | 3 |
| 8 | C | C | 2 | 2 |
| 9 | D | C | 1 | 2 |
| 10 | D | D | 3 | 2 |
| 11 | D | D | 3 | 3 |
| 12 | B | C | 1 | 1 |
| 13 | D | C | 2 | 2 |
| 14 | C | C | 2 | 2 |
| 15 | C | C | 3 | 3 |

The corresponding lower lid skin sites were then evaluated at 1 hour and at 3 hours following a polymer film application. The scale was used by the three trained observers to evaluate the improvement in the severity of the lower lid puffiness and wrinkles at each timepoint. These results are tabulated in Table 2 and Table 3, respectively.

TABLE 2
Change in Lower Lid Severity Grades following
1 hour of Polymer film application.

| | 1 hour | | | |
|---|---|---|---|---|
| | Average of | Average of | Average of | Average of |
| | Change from | Change from | Change from | Change from |
| | Baseline: L | Baseline R | Baseline: L | Baseline R |
| patient no. | Puffiness | Puffiness | Wrinkle | Wrinkle |
| 1 | −1 | −1 | −1 | −1 |
| 2 | −1 | −1 | −1 | 0 |
| 3 | −1 | −1 | 0 | 1 |
| 4 | 0 | −1 | −2 | −2 |
| 5 | 0 | 0 | −1 | −1 |
| 6 | 0 | 0 | −1 | −1 |
| 7 | 0 | 0 | −2 | −2 |
| 8 | 0 | 0 | −1 | −1 |
| 9 | 0 | 1 | 0 | −1 |
| 10 | −1 | −1 | −2 | 0 |
| 11 | 0 | 0 | −1 | −1 |
| 12 | 0 | −1 | 0 | 0 |
| 13 | −2 | 0 | −1 | −1 |
| 14 | −1 | −1 | −1 | −1 |
| 15 | 0 | 0 | −1 | −1 |

TABLE 3
Change in Lower Lid Severity Grades following
3 hours of Polymer film application.

| | 3 hours | | | |
|---|---|---|---|---|
| | Average of | Average of | Average of | Average of |
| | Change from | Change from | Change from | Change from |
| | Baseline: L | Baseline R | Baseline: L | Baseline R |
| Patient No. | Puffiness | Puffiness | Wrinkle | Wrinkle |
| 1 | −1 | −1 | −2 | −1 |
| 2 | −2 | −2 | −2 | −1 |
| 3 | −2 | −1 | 0 | −1 |
| 4 | −1 | −1 | −3 | −1 |
| 5 | −1 | 0 | −1 | 0 |
| 6 | 0 | 0 | −1 | 0 |
| 7 | 0 | 0 | −2 | 1 |
| 8 | 0 | 0 | −1 | 0 |
| 9 | 0 | 1 | 0 | 0 |
| 10 | −2 | −1 | −2 | 0 |
| 11 | −1 | 0 | −1 | −1 |
| 12 | NA | NA | NA | NA |
| 13 | NA | NA | NA | NA |
| 14 | −1 | −1 | −1 | 0 |
| 15 | −1 | −1 | −2 | −1 |

### EXAMPLE 2

A 62 year old woman was diagnosed with severe lower lid puffiness and severe wrinkles An ocularplastic surgeon eye-typed her left and right lower lid severities using the tool shown in FIG. 1 as 3D and 3D, respectively. The patient was seeking noninvasive intervention. A polymer film was prescribed to correct the puffiness and wrinkles by at least one grade. At three hours following polymer film application, the ocularplastic surgeon evaluated the patient again. Using the eye-typing tool, the treatment plan recommended yielded improved lower lid severity grades as indicated by the respective grades of 0C and 2C.

### EXAMPLE 3

A 40-year old patient with baseline grades of 2C and 2C (using the 16 cell grid scale as shown in FIG. 1) for her left and her right lower lid region. A variety of treatment plans were recommended by her physician, along with the anticipated outcomes. Fractional laser would provide an anticipated improvement in her wrinkles but not puffiness to yield grades of 1C and 1C. A lower lid blepharoplasty would yield an anticipated improvement in the puffiness reflecting grades of 2A and 2A. Topical tretinoin at 0.05%, used daily over the course of 1 year, would yield anticipated outcomes of 1C and 1C. A polymer film would provide an outcome of 1B and 1B.

### EXAMPLE 4

A polymer film was applied by a trained product applicator to the lower lids of each patient, following baseline photography and evaluations by the investigator. In total, 96 women, ages 30 to 77 were evaluated. Three hours after the polymer film application, each patient was evaluated again by the investigator and another set of photographs were taken. An assessment device of the present invention, the Biesman Anderson Gilchrest (B.A.G.) Lower Lid Typing Scale as shown in FIG. 5, was used by the investigators to evaluate the patients both before application of the polymer film and three hours post application. The B.A.G. scales is a 1-9 photonumeric scale that categorizes a range of 9 bag severities (i.e., under eye puffiness/swelling) ranging from mild to severe. Wrinkle severity was characterized using a four grade scale where grades of 1, 2, 3 and 4 correspond to none, mild, moderate and severe lower lid wrinkling, respectively.

A seven grade (−3 to 3) global aesthetic improvement scale (GAIS) was used at the end of three hours to describe the overall facial improvement resulting from polymer film application. GAIS is a standard scale used in the art. GAIS is a subjective scale based on the perceived improvement relative to baseline judged by an unbiased observer. GAIS values of 0, 1, 2 and 3 correspond to no improvement, visible improvement, marked improvement, and optimal cosmetic result. GAIS values or −1, −2, and −3 correspond to worse, much worse, and very much worse, respectively.

The grades for bags and wrinkles as determine by the B.A.G. scale and the grade based upon GAIS were entered into a spreadsheet and a pivot table function was utilized to sort through the performance data provided below.

Prior to application of the polymer film, 71% of the patients had moderate bags (B.A.G. grades 4, 5 and 6) and the total patient population spanned grades 3 to 9.

Three hours following application of polymer film, the patients were evaluated using the B.A.G. scale and the GAIS. As the B.A.G. provides for measurements of both under-eye bags and wrinkles, two grades are assigned per patient, one for under-eye bags and one for wrinkles GAIS provides only one measurement that characterizes the patient's overall appearance and is not specific to under-eye bags or wrinkles Also, in contrast to the B.A.G. scale, where the patient is graded using the scale shown in FIG. 5, the GAIS result is a subjective score determined by the investigator alone.

Using the B.A.G. scale, 93.75% of the patients demonstrated a 1 grade or more improvement in the appearance of bag severity on the 9 grade axis of the scale measuring puffiness. In particular, 22.39% improved 1 grade with regard to the under eye bags; 27.08% improved 2 grades; 21.88% improved 3 grades; and 22.4% improved 4 grades or better. 6.25% did not respond to the treatment (i.e., the change in BAG grade of 0 or less; or people not showing improvement). On average, a 2.4 grade (p<0.0001, student's t-test for statistical significance) improvement in the appearance of the under-eye bags was observed by the investigators.

Using the B.A.G. scale, 87.5% of the patients demonstrated a one grade or more improvement in the appearance of lower lid wrinkling, based upon the 4 grade axis of the scale measuring wrinkles In particular, 47.92% improved 1 grade with regard to wrinkles; 31.25% improved 2 grades; and 8.33% improved 3 grades. 12.5% did not respond to the treatment. On average, a 1.3 grade (p<0.0001, student's t-test for statistical significance) improvement in the appearance of lower lid wrinkles was observed.

Using the GAIS scale, 98.96% of the patients achieved GAIS of 1 or greater, with 83.33% showing marked to optimal improvements in global facial appearance. In particular, 15.63% showed visible improvement; 37.5% showed marked improvement; 45.83% showed optimal improvement; and 1.04% showed no improvement.

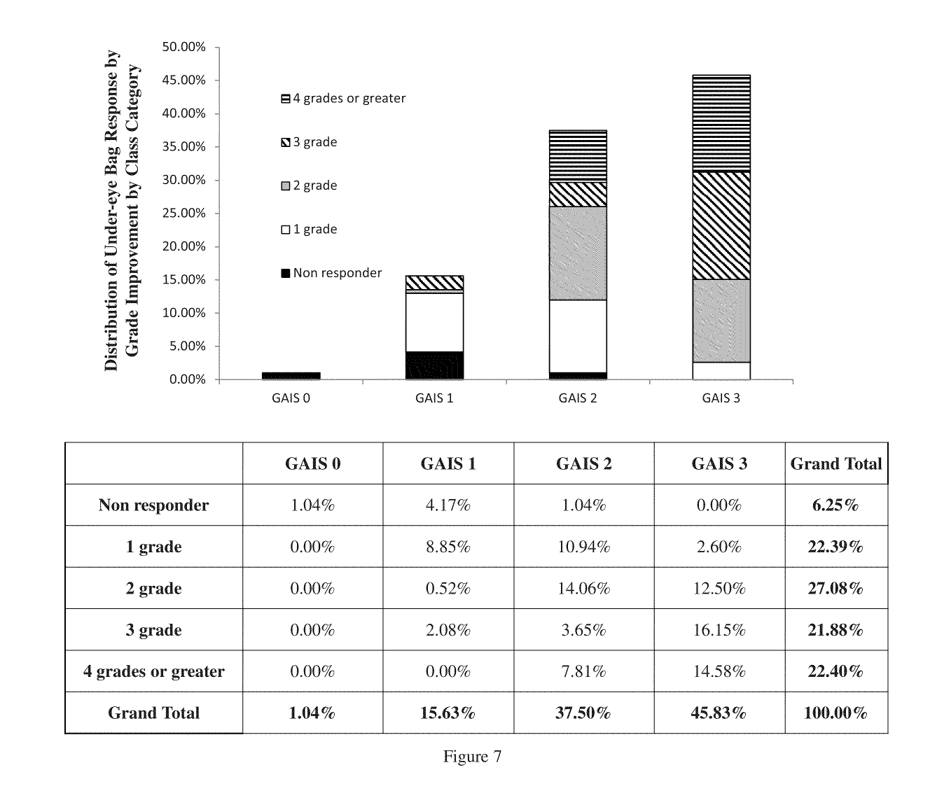

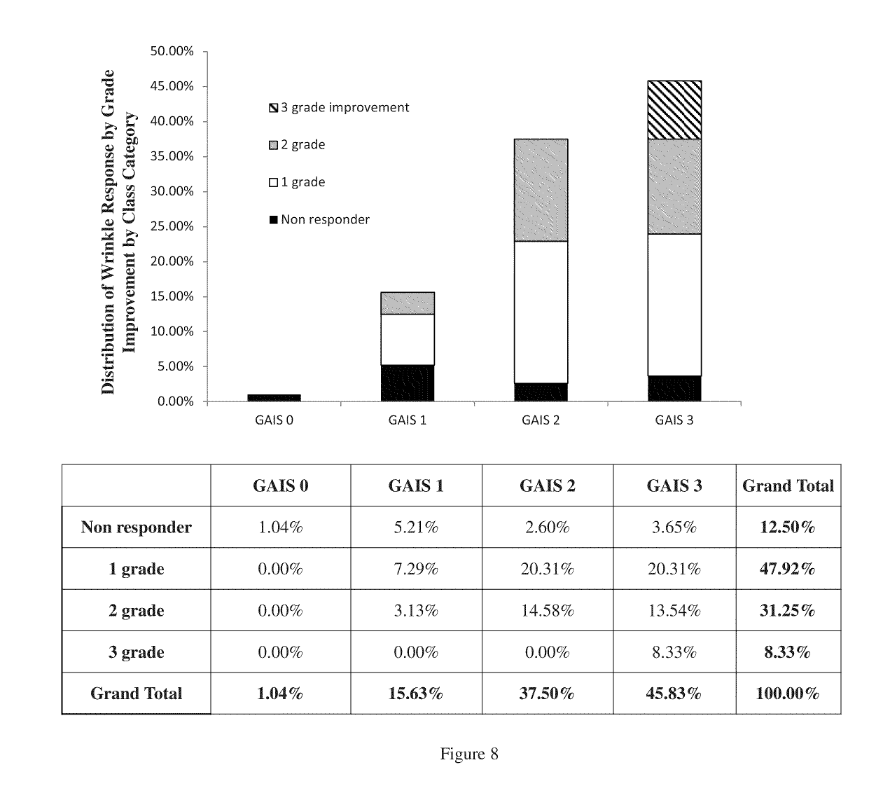

The results for the two scales are compared in FIGS. 7 and 8. In particular, the results from use of the B.A.G. scale to measure puffiness/swelling (i.e., under eye bags) is compared to the evaluation of the same patients with GAIS in FIG. 7. FIG. 8 compares the same GAIS scores to the B.A.G. score for the patient's wrinkles Patients demonstrating a one grade improvement in the appearance of bags or wrinkles using the B.A.G. scale were able to achieve GAIS of 1, 2 and 3. As a one grade improvement in the appearance of bags could result in a GAIS of 1, 2 or 3, the GAIS score only reflects an overall improvement of the facial appearance and does not necessary provide any information as to whether a particular treatment improved the severity of the under-eye bags specifically. Moreover, a GAIS score provides no specific information regarding improvement of either under-eye bags or wrinkles seperately. Comparing the results regarding the under-eye bag score from FIG. 7 to the wrinkle score from FIG. 8, it can be seen that for certain patients the improvement in under-eye bag and wrinkles is not commensurate. For example, for patients receiving a GAIS score of 0, 1, 2, or 3 saw varying grades of improvement depending on whether the patient's under-eye bags or wrinkles were measured. Accordingly, use of a scale such as GAIS cannot differentiate between improvement in a particular facial feature such as under eye-bags (or wrinkles) with improvement overall. In contrast, the B.A.G. scale provides two distinct readings, in this case, under-eye bags and wrinkles, and accordingly, the BAG scale provides a more precise diagnosis as compared to the GAIS output. Moreover, the B.A.G. scale is absolute and requires no outside reference for grading and focused at the target site.

Thus the several aforementioned objects and advantages are most effectively attained. Although preferred embodiments of the invention have been disclosed and described in detail herein, it should be understood that this invention is in no sense limited thereby and its scope is to be determined by that of the appended claims.

What is claimed is:

1. An assessment device for quantifying severity of conditions associated with human facial wrinkles and swelling and for determining a treatment plan or treatment course, the assessment device comprising:
a data collection of at least two coordinates, including at least a first coordinate and a second coordinate;
the first coordinate including a plurality of increments of degree of wrinkle severity;
the second coordinate including a plurality of increments of degree of facial swelling;
the data collection including a plurality of data spaces, wherein a data space is formed for each combination of one of the plurality of increments of degree of wrinkle severity and one of the plurality of increments of degree of facial swelling; and
each of the data spaces including data which corresponds to a treatment plan or treatment course for the combination of wrinkle severity and facial swelling associated with the data space.

2. The assessment device of claim 1 wherein the first coordinate includes a plurality of descriptions of the increments of degree of wrinkle severity.

3. The assessment device of claim 2 wherein the second coordinate includes a plurality of descriptions of the increments of degree of facial swelling.

4. The assessment device of claim 3 further including depictions corresponding to each increment of wrinkle severity and each increment of facial swelling.

5. The assessment device of claim 4 wherein the depictions are photographs.

6. The assessment device of claim 4 wherein at least one of the increments of degree of facial swelling includes a plurality of sub-increments, wherein each of the sub-increments includes a description of the sub-increment of facial swelling, and wherein each combination of a sub-increment of degree of facial swelling and increment of degree of wrinkle severity corresponds to one of the data spaces.

7.-10. (canceled)

11. A method of treating wrinkles and facial swelling including the steps of:
providing assessment device of claim 1, which includes a plurality of data spaces which correspond to treatment plans or treatment course for combinations of degree of wrinkle severity and degree of facial swelling associated with the data space;
using the assessment device to determine a degree of wrinkle severity and a degree of facial swelling of a patient; and
applying a treatment plan or treatment course to the patient, wherein the treatment plan or treatment course corresponds to the data space which corresponds to the determined degree of wrinkle severity and determined degree of facial swelling of the patient.

12. The method of claim 11 wherein the first coordinate includes a plurality of descriptions of the increments of degree of wrinkle severity.

13. The method of claim 12 wherein the second coordinate includes a plurality of descriptions of the increments of degree of facial swelling.

14. The method of claim 13 further including depictions corresponding to each increment of wrinkle severity and each increment of facial swelling.

15. The method of claim 14 wherein the depictions are photographs.

16.-20. (canceled)

21. The assessment device of claim 1 wherein the treatment plans or treatment courses further include data as to at least one expected outcome of the treatment plan or treatment course.

22. (canceled)

23. An assessment device for quantifying severity of conditions associated with human facial swelling and for determining a treatment plan or treatment course, the assessment device comprising:
a plurality of increments of degree of facial swelling; wherein a data space is formed for the plurality of increments of degree of facial swelling; and
each of the data spaces including data which corresponds to a treatment plan or treatment course for the facial swelling associated with the data space.

24. The assessment device of claim 23 further including depictions corresponding to each increment of facial swelling.

25. The assessment device of claim 24 wherein the depictions are photographs.

26. The assessment device of claim 24 wherein at least one of the increments of degree of facial swelling includes a plurality of sub-increments, wherein each of the sub-increments includes a description of the sub-increment of facial swelling, and wherein each combination of a sub-increment of degree of facial swelling corresponds to one of the data spaces.

27.-30. (canceled)

31. A method of treating facial swelling including the steps of:
providing an assessment device which includes a plurality of data spaces which correspond to treatment plans or treatment course for degree of facial swelling associated with the data space;
using the assessment device to determine a degree of facial swelling of a patient; and
applying a treatment plan or treatment course to the patient, wherein the treatment plan or treatment course corresponds to the data space which corresponds to the determined degree of facial swelling of the patient;
wherein the assessment device comprises:
a plurality of increments of degree of facial swelling, wherein a data space is formed for the plurality of increments of degree of facial swelling.

32. The method of claim 31 further including depictions corresponding to each increment of each increment of facial swelling.

33. The method of claim 32 wherein the depictions are photographs.

34.-38. (canceled)

39. The assessment device of claim 31 wherein the treatment plans or treatment courses further include data as to at least one expected outcome of the treatment plan or treatment course.

40. (canceled)
